(19) United States
(12) Patent Application Publication
(10) Pub. No.: US 2019/0209390 A1
(43) Pub. Date: Jul. 11, 2019

(54) NOSE CLEANING SWAB SYSTEM

(71) Applicant: Domenic Silvaroli, Toronto (CA)

(72) Inventor: Domenic Silvaroli, Toronto (CA)

(21) Appl. No.: 15/866,934

(22) Filed: Jan. 10, 2018

(51) Int. Cl. A61F 13/38

(52) U.S. Cl. CPC A61F 13/38; A61B 2017/246; A61M 2210/0618

(57) ABSTRACT

A nose cleaning swab system including a cleaning swab having a handle and an auger swab head. The auger swab head has a swirled profile including a plurality of spaced ribs. As a user twists the handle, the auger swab head efficiently removes material out of a bodily orifice when the auger swab head is inserted into the bodily orifice such as a nose. The auger swab head is tapered from a tip to a base where the auger swab head is connected to the handle creating a continuous one-piece the cleaning swab.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of cleaning tools and more specifically relates to swabs.

### 2. Description of Related Art

Currently there are a number of solutions for nostril cleansing. Some of these solutions attempt to clean the inside of the nose, but these solutions fail to meet the needs of the market because the heads of the device need to be constantly cleaned and sanitized for later re-use. Other solutions attempt to remove mucous from the nostrils, but these solutions are similarly unable to meet the needs of the market because it leaves other remaining debris in the nose. Still other solutions seek to use disposable material to clean nostrils, but these solutions also fail to meet market needs because it prevents a user from cleaning nostrils multiple times with the same device before having to dispose of the cleaner. Therefore, there currently exists a need in the market for an apparatus that is a re-usable nostril cleanser that is easy to use and hygienic and effectively clears debris from the user's nose.

U.S. Pat. No. 9,532,793 to Paul David Lincoln Wilson relates to a nose comforter and cleaning device. The described nose comforter and cleaning device includes disposable hygienic nostril cleansing tool is described that consists of a dual-purpose head (cotton swab and scraper tip), flexible shaft, and cotton debris cleaner in the tool body.

### BRIEF SUMMARY OF THE INVENTION

In view of the foregoing disadvantages inherent in the known cleaning tools art, the present disclosure provides a novel nose cleaning swab system. The general purpose of the present disclosure, which will be described subsequently in greater detail, is to provide an auger swab head to clean a bodily orifice when the auger swab head is inserted into the bodily orifice and removed therefrom.

A nose cleaning swab system is disclosed herein. The nose cleaning swab system includes a cleaning swab having a handle and an auger swab head (having a flighting). The auger swab head is positioned on a distal end of the handle. The cleaning swab is manually operated by the user twisting the handle. The auger swab head comprises a swirled profile having a plurality of spaced ribs (flighting). As a user twists the handle, the auger swab head efficiently removes material out of a bodily orifice when the auger swab head is inserted into the bodily orifice such as a nose or ear. The auger swab head is tapered from a tip to a base where the auger swab head is connected to the handle creating a continuous one-piece cleaning swab. The handle is configured to be gripped by a hand of a user and twist the handle and the auger swab head to auger material out of a bodily orifice (manually as rotated) when the auger swab head is inserted into the bodily orifice such as a nose or ear. Nose Swab presented herein is a standard size for the average nostril size, for example an adult. It may be offered in different sizes for infants/children nostrils (possibly in the sizes of small/medium/large swab sizes).

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a nose cleaning swab system, constructed and operative according to the teachings of the present disclosure.

FIG. 1 is a perspective view of the nose cleaning swab system during an ‘in-use’ condition, according to an embodiment of the disclosure.

FIG. 2 is a perspective view of the nose cleaning swab system of FIG. 1, according to an embodiment of the present disclosure.

FIG. 3 is a perspective view of the nose cleaning swab system of FIG. 1, according to an embodiment of the present disclosure.

FIG. 4 is a perspective view of the nose cleaning swab system of FIG. 1, according to an embodiment of the present disclosure.

FIG. 5 is a perspective view of the nose cleaning swab system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to cleaning tools and more particularly to a nose cleaning swab system as used to improve the cleaning of a user's nose using an auger like cotton swab formed as a functional flighting.

Generally, the invention includes a tip shaped with a slight swirl. The whole nose cleaning swab is made out of dental cotton material or other suitable material. A user may simply insert the device, twist, and clean one's nose or other bodily orifice.

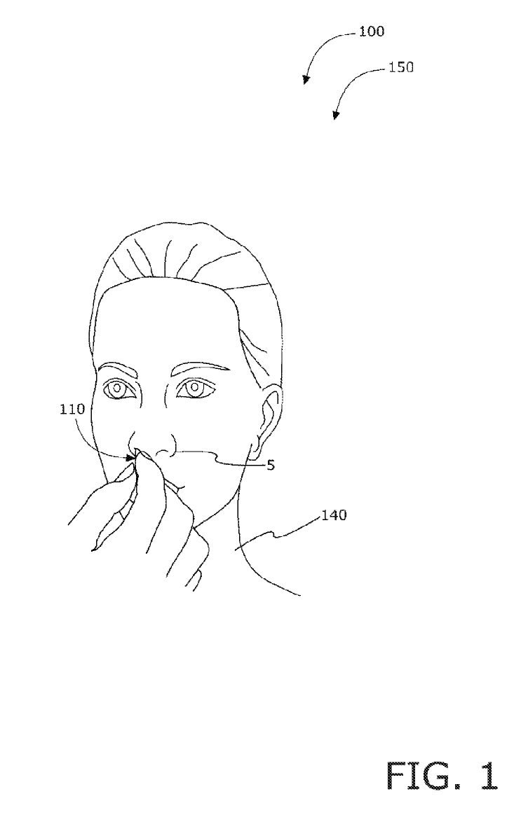

Referring now more specifically to the drawings by numerals of reference, there is shown in FIGS. 1-5, various views of a nose cleaning swab system 100. FIG. 1 shows a nose cleaning swab system 100 during an ‘in-use’ condition 150, according to an embodiment of the present disclosure. As illustrated, the nose cleaning swab system 100 may include a cleaning swab 110 having a handle 120 and an auger swab head 130. The handle 120 is configured to be gripped by a hand of a user 140 and twist the handle 120 and the auger swab head 130 to auger material out of a bodily orifice 5 when the auger swab head 130 is inserted into the bodily orifice 5 such as a nose or ear. Nose cleaning swab system 100 provides a portable solution for nose hygiene.

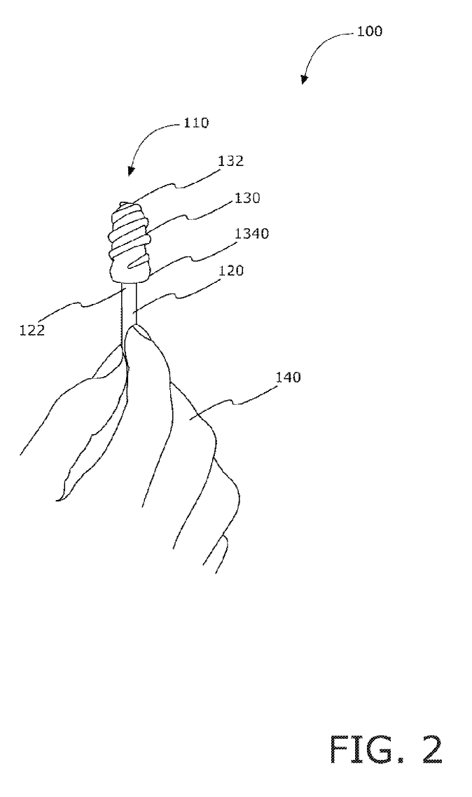

Referring now to FIG. 2 showing a perspective view of the nose cleaning swab system 100 of FIG. 1, according to an embodiment of the present disclosure. As above, the nose cleaning swab system 100 may include the cleaning swab 110 having the handle 120 and the auger swab head 130 for use in cleaning a user's nose. The auger swab head 130 is positioned on a distal end 122 of the handle 120. The auger swab head 130 is tapered from a tip 132 towards a base 134 where the auger swab head 130 is connected to the handle 120 creating a continuous one-piece cleaning swab 110. By rotating the handle 120 and the cleaning swab 110 appropriately, material will be withdrawn from the nose and provide cleaning action. The device provides an efficient cleaning device for maintaining nose hygiene.

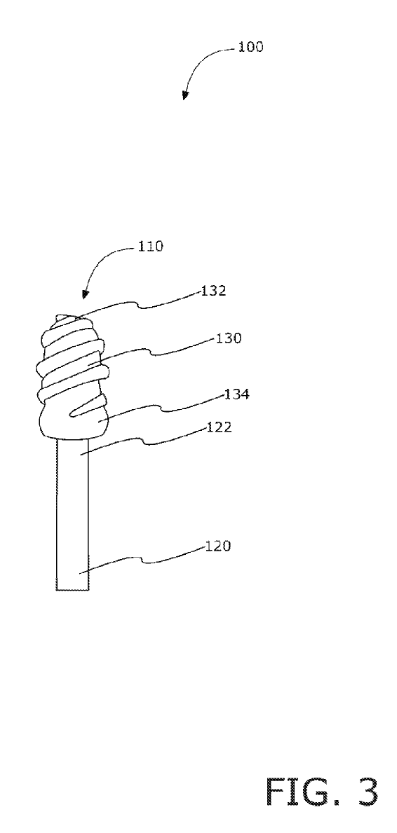

FIG. 3 shows a perspective view of the nose cleaning swab system 100 of FIG. 1, according to an embodiment of the present disclosure. As above, the nose cleaning swab system 100 may include the cleaning swab 110 having the handle 120 and the auger swab head 130. The handle 120 is denser than the auger swab head 130 allowing a user to manipulate the device using the handle 120 for support. The handle 120 is semi-rigid and the auger swab head 130 is semi-flexible. The auger swab head 130 comprises an absorbent material configured to absorb the material out of the bodily orifice 5. The absorbent material may include cotton or other suitable absorbent material. The device is soft to touch for user comfort when cleaning one's nose.

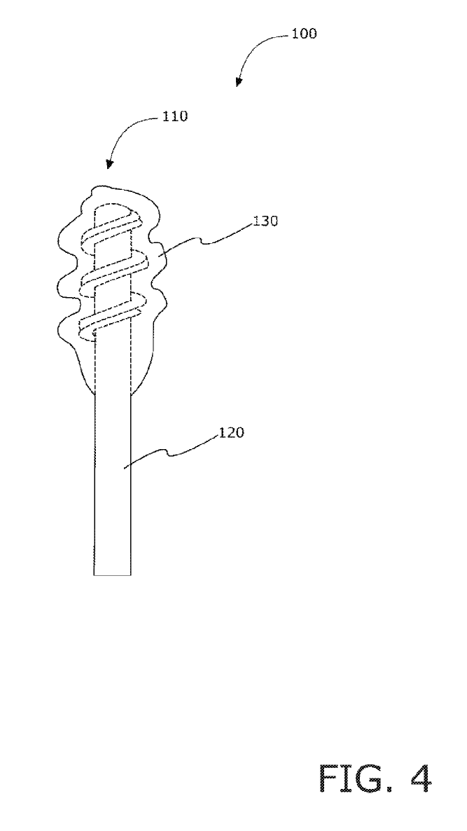

Referring now to FIG. 4 showing a perspective view of the nose cleaning swab system 100 of FIG. 1, according to an embodiment of the present disclosure. As above, the nose cleaning swab system 100 may include the cleaning swab 110 having the handle 120 and the auger swab head 130 in functional combination. In a preferred embodiment, the auger swab head 130 comprises a length of approximately ¾-inch from the tip 132 to the base 134. The handle 120 comprises a length of approximately 0.75 of an inch. The handle 120 comprises a width of approximately 0.375 of an inch. The cleaning swab 110 comprises a total length of approximately 1.5 inches. The tip 132 of the auger swab head 130 has a smaller width than the base 134 of the auger swab head 130. The tapered profile allows the device to fit within a nose.

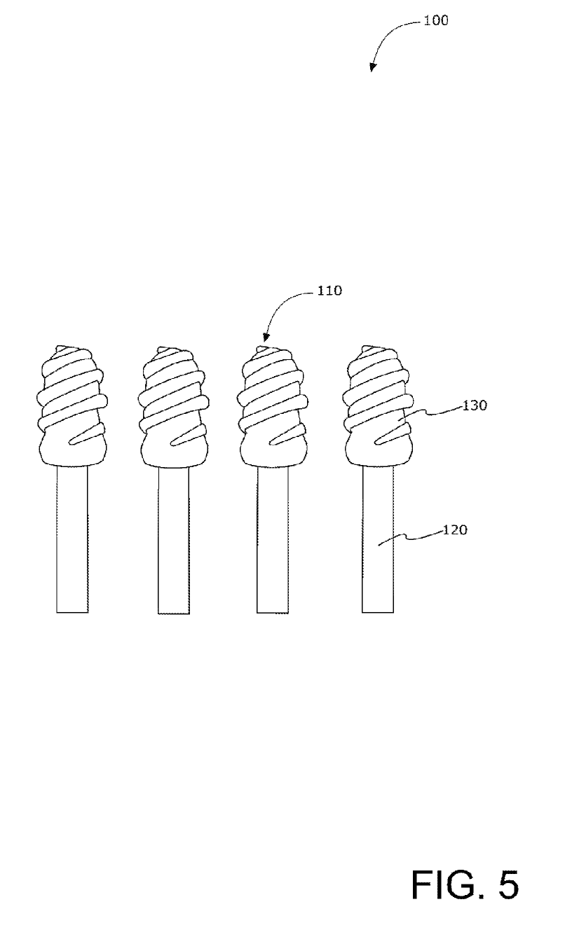

FIG. 5 shows a perspective view of the nose cleaning swab system 100 of FIG. 1, according to an embodiment of the present disclosure. As above, the nose cleaning swab system 100 may include the cleaning swab 110 having the handle 120 and the auger swab head 130. The cleaning swab 110 is preferably disposable but other embodiments may be provided. The cleaning swab 110 is manually operated by the user 140 twisting the handle 120. The auger swab head 130 comprises a swirled profile having a plurality of spaced ribs. As a user 140 twists the handle 120, the auger swab head 130 efficiently removes material out of a bodily orifice 5 when the auger swab head 130 is inserted into the bodily orifice 5 such as a nose or ear. The auger swab head 130 has a diameter that is approximately equal to a diameter of a nostril. In certain embodiments, the nose cleaning swab system 100 further comprises a form-fitting jacket for the handle 120. The form-fitting jacket may be clear plastic, colored cardboard or paper material.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A nose cleaning swab system comprising:
a cleaning swab having;
a handle; and
an auger swab head;
wherein said auger swab head is positioned on a distal end of said handle;
wherein said auger swab head is tapered from a tip to a base where said auger swab head is connected to said handle creating a continuous one-piece said cleaning swab; and
wherein said handle is configured to be gripped by a hand of a user and twisted via said handle and said auger swab head to auger material out of a bodily orifice via a flighting when said auger swab head is inserted into said bodily orifice.

2. The nose cleaning swab system of claim 1, wherein said handle is denser than said auger swab head.

3. The nose cleaning swab system of claim 2, wherein said handle is semi-rigid.

4. The nose cleaning swab system of claim 2, wherein said auger swab head is semi-flexible.

5. The nose cleaning swab system of claim 1, wherein said bodily orifice is a nose.

6. The nose cleaning swab system of claim 1, wherein said auger swab head comprises an absorbent material configured to absorb said material.

7. The nose cleaning swab system of claim 6, wherein said absorbent material of said auger swab head comprises cotton.

8. The nose cleaning swab system of claim 1, wherein said auger swab head comprises a length of approximately ¾-inch from said tip to said base.

9. The nose cleaning swab system of claim 8, wherein said handle comprises a length of approximately 0.75 of an inch.

10. The nose cleaning swab system of claim 9, wherein said handle comprises a width of approximately 0.375 of an inch.

11. The nose cleaning swab system of claim 10, wherein said cleaning swab comprises a total length of approximately 1.5 inches.

12. The nose cleaning swab system of claim 1, wherein said tip of said auger swab head has a smaller width than said base of said auger swab head.

13. The nose cleaning swab system of claim 1, wherein said cleaning swab is disposable.

14. The nose cleaning swab system of claim 1, wherein said cleaning swab is manually operated by said user twisting said handle.

15. The nose cleaning swab system of claim 1, wherein said auger swab head has a diameter that is approximately equal to a diameter of a nostril.

16. The nose cleaning swab system of claim 1, wherein said auger swab head comprises a swirled profile having a plurality of spaced ribs.

17. The nose cleaning swab system of claim 1, further comprises a form-fitting jacket for said handle.

18. A nose cleaning swab system, the nose cleaning swab system comprising:
a cleaning swab having;
a handle; and
an auger swab head having a flighting;
wherein said auger swab head is positioned on a distal end of said handle;
wherein said auger swab head is tapered from a tip to a base where said auger swab head is connected to said handle creating a continuous one-piece said cleaning swab;
wherein said handle is denser than said auger swab head;
wherein said handle is semi-rigid;
wherein said auger swab head is semi-flexible;
wherein said handle is configured to be gripped by a hand of a user and twist said handle and said auger swab head to auger material out of a bodily orifice when said auger swab head is inserted into said bodily orifice;
wherein said bodily orifice is a nose;
wherein said auger swab head comprises an absorbent material configured to absorb said material;
wherein said absorbent material of said auger swab head comprises cotton;
wherein said auger swab head comprises a length of approximately ¾-inch from said tip to said base;
wherein said handle comprises a length of approximately 0.75 of an inch;
wherein said handle comprises a width of approximately 0.375 of an inch;
wherein said cleaning swab comprises a total length of approximately 1.5 inches;
wherein said tip of said auger swab head has a smaller width than said base of said auger swab head;
wherein said cleaning swab is disposable;
wherein said cleaning swab is manually operated by said user twisting said handle;
wherein said auger swab head has a diameter that is approximately equal to a diameter of a nostril; and
wherein said auger swab head comprises a swirled profile having a plurality of spaced ribs as said flighting.
